(19) United States
(12) Patent Application Publication
(10) Pub. No.: US 2009/0048187 A1
(43) Pub. Date: Feb. 19, 2009

(54) Chemopreventive, Anticancer and Anti-Inflammatory Effects of Pinoresinol-Rich Olives

(76) Inventors: Luigi Ricciardiello, Dallas, TX (US); Clement Richard Boland, Dallas, TX (US); Marco Romano, Napoli (IT); Vincenzo Fogliano, Rome (IT)

Correspondence Address: CHALKER FLORES, LLP, 2711 LBJ FRWY, Suite 1036, DALLAS, TX, 75234, US

(21) Appl. No.: 12/182,101

(22) Filed: Jul. 29, 2008

Related U.S. Application Data
(60) Provisional application No. 60/952,801, filed on Jul. 30, 2007.

(51) Int. Cl. A61K 31/7028; C12N 5/08; A61K 31/22; A61K 31/506; A61P 35/00; A61K 31/538; A61K 31/407; A61K 31/05

(52) U.S. Cl. 514/34; 435/377; 514/549; 514/731; 514/410; 514/256; 514/229.8

(57) ABSTRACT

The present invention relates to a composition and method for preventing and treating cancer and modulating cell proliferation using compositions extracted from pinoresinol-rich Olea europaea Caiazzana olives. The present invention provides contacting one or more cells with a pharmaceutical effective amount of a polyphenolic composition isolated from Olea europaea Caiazzana olives. The polyphenolic composition includes one or more polyphenolic compounds extracted from Olea europaea Caiazzana olives.

### CROSS-REFERENCE TO RELATED APPLICATIONS

This application claims priority to U.S. Provisional Application Ser. No. 60/952,801 filed on Jul. 30, 2007, the entire contents of which are incorporated herein by reference.

### STATEMENT OF FEDERALLY FUNDED RESEARCH

This invention was made with U.S. Government support under Contract No. R01 CA72851 and R01 CA98572 awarded by the National Cancer Institute of the NIH. The government has certain rights in this invention.

### TECHNICAL FIELD OF THE INVENTION

The present invention relates in general to the field of cancer treatment and prevention, specifically methods for the preparation and use of formulations of isolated and purified pinoresinol extracts derived from extra virgin olive oil for the treatment and/or prevention of cancer.

### BACKGROUND OF THE INVENTION

Without limiting the scope of the invention, its background is described in connection with methods for the preparation, use and formulations containing polyphenol derived from olives and in particular, lignan compounds extracted from extra virgin olive oil.

Colorectal cancer is the fourth commonest cancer in western countries, affecting over one million of new cases globally every year, with nearly 500,000 deaths. The occurrence rate of colorectal cancer is dependent on many factors including family history and diet. In the United States and many other developed countries a diet low in fiber and high in fat is common; however, countries having diets that are high in fiber and low in fat have show fewer instances of colorectal cancer in individuals. Although colorectal cancer is treatable if discovered early, there are limited methods of detection available. Numerous research programs have been devised to tackle colorectal cancer, and compound such as polyphenol have been widely investigated for their anticancer properties in a variety of tumors and models.

For example, U.S. Pat. No. 7,192,612 provides methods and compositions of preventing or treating cancer by the administration of a combination of therapeutically amount of catechins, a group of polyphenols found in green tea, and Capsicum extracts. The compositions contain various combinations of the catechins and Capsicum extracts, in combination with each other or other therapeutic agents and are used to treat primary and metastatic cancers in humans.

Another example includes U.S. Pat. No. 6,096,359 which provides preparation of polyphenol fractions of Camellia sinensis (tea), use thereof and formulations containing them. The polyphenol fractions are extracts deprived of caffeine but containing the polyphenols deriving from epigallocatechin in a natural ratio. The use of these extracts, alone or in combination with other active principles, is of interest to the food, pharmaceutical, and cosmetic industry, especially to treat cytotoxic and oxidative conditions.

However, the anticancer effects of polyphenol have been shown to be compound-dependent and dependent on the size of the molecule and functional group, shape of the molecule and functional group, number of functional groups and type of functional groups; in addition, the effect of the compounds range from apoptosis, induction of cell cycle arrest, decrease in cell proliferation and modulation of epigenetic changes. Approximately 8,000 identified phenolic compounds have been identified but the full extent of phenolic compounds is yet to be determined. To complicate further the identification and characterization of phenolic compounds, the polyphenolic compound family, the specific polyphenolic compounds present in individual genius and species, as well as variations of compounds as a result of geographical and regional variations has yet to be characterized. Given the compound-specific dependent nature of these polyphenolic compounds, it remains a challenge in this field of art to identify novel chemopreventive candidates and demonstrate its anticancer properties.

### SUMMARY OF THE INVENTION

The present inventors recognized that diets rich in fruits, vegetable, olive oil and red wine, were associated with a lower incidence of cancer due to the elevated amounts of phenolic compounds in those foods. The phenolic compounds have a broad spectrum of properties including antineoplastic, antioxidant and anti-inflammatory. However, in addition to its unsaturated fatty acids, olive oil is the source of several micronutrient components with beneficial properties, such as α-tocopherol, carotenoids, sterols and phenolic compounds, and particularly, phenolic fraction from two different varieties of olive grown in south-central Italy.

The two varieties of olive have significantly different phenolic compositions: pinoresinol is the major phenolic fraction of the Caiazzana olives grown from the Campania region of southern Italy, and oleocanthal is the main component of Ravece olives grown in the same region of Italy. The present inventors recognize that these pinoresinol-rich extracts are able to decrease cell viability, induce apoptosis and modulate cell cycle dynamics. More importantly, the present invention provides phenolic compounds of extracts from extra virgin olive oil that have an anticancer effect.

The present invention relates to a composition and method for preventing and treating cancer using pinoresinol-rich extra virgin olive oil. The present inventors recognize pinoresinol-rich extract is able to decrease cell viability, induce apoptosis and modulate cell cycle dynamics in cancer cells. More importantly, the present invention demonstrates that pinoresinol-enriched extracts from extra virgin olive oil have potent chemopreventive properties against cancer cells at very low concentrations.

In one embodiment, Caiazzana olives from Italy were harvested in November 2005 and within 4 hours, extracted in a continuous extraction plant, using a percolation-centrifugation system with hammer crushers, a centrifugal decanter and a separator. Unfiltered oils were stored in filled amber glass bottles at room temperature until extraction. In this embodiment, the present invention demonstrates the anticancer effects of phenolic compounds contained in extra virgin olive oil on in vitro models of colorectal cancer. Activities of extra virgin olive oil extracts on cell proliferation, apoptosis, and cell cycle regulation, and specific pathways involved in the regulation of cell growth are provided herein.

The present invention provides a method of modulating cell proliferation by contacting one or more cells with a pharmaceutical effective amount of a polyphenolic composition. The polyphenolic composition includes one or more polyphenolic compounds extracted from one or more plant tissues, wherein the one or more polyphenolic compounds affects cell proliferation

The present invention also provides a polyphenolic composition for the treatment or prevention of colorectal cancer in a subject. The polyphenolic composition includes a pharmaceutical carrier and an pharmaceutical effective amount one or more polyphenolic compounds extracted from one or more tissues of an olive or olive oil and affect one or more colorectal cancer cells. The one or more polyphenolic compounds include pinoresinol, oleocanthal, hydrotyrosol, tyrosol, secoiridoids, lignans, gallic acid, resveratrol, hydroxytirosol, deacetoxy-ligstroside aglycone, p-HPEA-EDA, 3,4-DHPEA-EA, p-HPEA-EA or mixtures thereof.

A method of anticancer therapy is also provided by the present invention. The therapy includes administering to a patient in need of anticancer therapy a pharmaceutical effective amount of a polyphenolic composition comprising one or more polyphenolic compounds extracted from one or more plant tissues. The one or more polyphenolic compounds affects the proliferation of one or more cells.

A pharmaceutical composition for the treatment of neoplasia in a subject. The composition includes a pharmaceutical carrier and a pharmaceutical effective amount of one or more polyphenolic compounds extracted from one or more olives. The polyphenolic composition affects abnormal cell proliferation.

A method for treating neoplasia in a subject by administering a pharmaceutical effective amount of a polyphenolic composition to the subject to treat a neoplasia is provided by the present invention. The polyphenolic composition comprises one or more polyphenolic compounds extracted from one or more Caiazzana olives from the Campania region of Italy and affects abnormal cell proliferation in the subject.

A method and composition for treating gastrointestinal inflammation in a subject by administering a pharmaceutical effective amount of a polyphenolic composition to the subject to treat gastrointestinal inflammation is provided by the present invention. Gastrointestinal inflammation may include, but not limited to, chronic gastrointestinal inflammation, and inflammatory bowel disease such as Crohn's disease and ulcerative colitis

The present invention provides a dietary supplement for the treatment or prevention of neoplasia in a subject. The supplement includes a pharmaceutical carrier and a pharmaceutical effective amount of one or more polyphenolic compounds extracted from one or more Caiazzana olives from the Campania region of Italy.

A kit for carrying out the therapeutic regimens of the invention comprise in one or more containers having therapeutically effective amounts of extra virgin olive oil polyphenol extract complexes in pharmaceutically acceptable form. The extra virgin olive oil polyphenol extract complex in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further includes in a container a pharmaceutically acceptable solution (e.g., saline, dextrose solution, etc.), preferably sterile, to reconstitute the complex to form a solution for injection purposes. The kit may also include a needle or syringe, preferably packaged in sterile form, for injecting said extra virgin olive oil polyphenol extracts, and/or a packaged alcohol pad.

The present invention also includes a method of anticancer therapy an individual at risk of developing a tumor a therapeutically effective amount of a composition of about 10 to about 30 percent by weight of an extract from Caiazzana olives from the Campania region of Italy.

The present invention includes a method of inducing apoptosis by administering a pharmaceutical effective amount of a polyphenolic composition to the subject. The polyphenolic composition comprises one or more polyphenolic compounds extracted from one or more Caiazzana olives from the Campania region of Italy and affects abnormal cell proliferation in the subject.

The present invention includes a method of inducing expression of one or more tumor suppressor genes by administering a pharmaceutical effective amount of a polyphenolic composition to the subject. The polyphenolic composition comprises one or more polyphenolic compounds extracted from one or more Caiazzana olives from the Campania region of Italy and affects abnormal cell proliferation in the subject.

The polyphenolic composition contains one or more polyphenolic compounds extracted from one or more Caiazzana olives from the Campania region of Italy and affects abnormal cell proliferation in a subject.

### BRIEF DESCRIPTION OF THE DRAWINGS

For a more complete understanding of the features and advantages of the present invention, reference is now made to the detailed description of the invention along with the accompanying figures and in which:

FIG. 1A is a graph of cell viability in RKO and SW480 cell lines;

FIG. 1B is a graph of cell viability after treatment with polyphenolic extracts from olive oil;

FIG. 2A is an image illustrating apoptotic response of cells to treatments with polyphenolic extracts from olive oil;

FIG. 2B is a graph demonstrates changes in the transcript levels of the pro-apoptotic gene Bax using real-time PCR;

FIG. 3 is a plot of the representative flow cytometry cell cycle profiles illustrating cell cycle analysis in samples treated with and without polyphenolic extracts from olive oil;

FIG. 4 is an image of a Western blot analysis of p53 and p53-regulated proteins with and without treatment of polyphenolic extracts from olive oil; and

FIG. 5 is an image of a Western blot that illustrates cell cycle checkpoint regulatory with and without treatment of polyphenolic extracts from olive oil.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “carrier” is used to describe a substance, whether biodegradable or not, that is physiologically acceptable for human or animal use and may be pharmacologically active or inactive.

The term “immediate release” as used herein is used to describe a release profile to effect delivery of an active as soon as possible, that is, as soon as practically made available to an animal, whether in active form, as a precursor and/or as a metabolite. Immediate release may also be defined functionally as the release of over 80 to 90 percent (%) of the active ingredient within about 60, 90, 100 or 120 minutes or less. Immediate release as used herein may also be defined as making the active ingredient available to the patient or subject regardless of uptake, as some actives may never be absorbed by the animal. Immediate release formulations of the active on a carrier, such as rolled or compressed beads, may be formulated such that the surface area is maximized on beads and the active is exposed immediately. The immediate release formulations may also include effervescing agents that cause the disintegration of the structure integrity of the active and carrier such that release of the active is maximized. Various immediate release dosage forms may be designed readily by one of skill in the art to achieve drug delivery to the stomach and small intestine, depending upon the choice of compression, adhesive materials and/or beading.

The terms “extended release” and “delayed release” as used herein is used to define a release profile to effect delivery of an active over an extended period of time, defined herein as being between about 60 minutes and about 2, 4, 6 or even 8 hours. Extended release may also be defined functionally as the release of over 80 to 90 percent (%) of the active ingredient after about 60 minutes and about 2, 4, 6 or even 8 hours. Extended release as used herein may also be defined as making the active ingredient available to the patient or subject regardless of uptake, as some actives may never be absorbed by the animal. Various extended release dosage forms may be designed readily by one of skill in art as disclosed herein to achieve delivery to both the small and large intestines, to only the small intestine, or to only the large intestine, depending upon the choice of coating materials and/or coating thickness.

“Extended release” and “delayed release” formulations may be prepared and delivered so that release is accomplished at some generally predictable location in the lower intestinal tract more distal to that, which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients, and some of their properties, include, but are not limited to: shellac, also called purified lac, a refined product obtained from the resinous secretion of an insect. This coating dissolves in media of pH>7.

The present pharmaceutical composition may also be provided in a variety of dosage forms, e.g., enveloped pharmaceutical, solution, suspension, cream, ointment, lotion, capsule, caplet, softgel, gelcap, elixir, syrup, emulsion, granule, gum, insert, jelly, paste, pastille, pellet, spray, lozenge, disk, magma, poultice, or wafer and the like. As used herein, the term “enveloped pharmaceutical” means a capsule, a suppository, a gel cap, a softgel, a lozenge, a sachet or even a fast dissolving wafer. The polyphenolic composition may be in the form of an immediate release, extended release or delayed release.

The term “modulate” as used herein, refers to a change or an alteration in the biological activity and may be an increase or a decrease in activity or any other change in the biological, functional, or immunological properties.

As used herein, the term “antioxidant” is intended to mean an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and the like.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and the like.

As used herein, the term “colorant” is intended to mean a compound used to impart color to liquid and solid (e.g., tablets and capsules) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red and the like.

As used herein, the term “flavorant” is intended to mean a compound used to impart a pleasant flavor and often odor to a pharmaceutical preparation. In addition to the natural flavorants, many synthetic flavorants are also used. Such compounds include, by way of example and without limitation, anise oil, cinnamon oil, cocoa, menthol, orange oil, peppermint oil and vanillin and the like.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and the like.

As used herein, the term “tablet antiadherents” is intended to mean agents which prevent the sticking of table formulation ingredients to punches and dies in a tableting machine during production. Such compounds include, by way of example and without limitation, magnesium stearate, talc, and the like.

As used herein, the term “tablet binders” is intended to mean substances used to cause adhesion of powder particles in table granulations. Such compounds include, by way of example and without limitation, acacia, alginic acid, carboxymethyl cellulose, sodium, compressible sugar ethylcellulose, gelatin, liquid glucose, methylcellulose, povidone and pregelatinized starch and the like.

As used herein, the term “tablet and capsule diluent” is intended to mean inert substances used as fillers to create the desired bulk, flow properties, and compression characteristics in the preparation of tablets and capsules. Such compounds include, by way of example and without limitation, dibasic calcium phosphate, kaolin, lactose, mannitol, microcrystalline cellulose, powdered cellulose, precipitated calcium carbonate, sorbitol, and starch and the like.

As used herein, the term “tablet direct compression excipient” is intended to mean a compound used in direct compression tablet formulations. Such compounds include, by way of example and without limitation, dibasic calcium phosphate and the like.

As used herein, the term “tablet disintegrant” is intended to mean a compound used in solid dosage forms to promote the disruption of the solid mass into smaller particles, which are more readily dispersed or dissolved. Such compounds include, by way of example and without limitation, alginic acid, carboxymethylcellulose, calcium, microcrystalline cellulose, polacrilin potassium, sodium alginate, sodium starch glycolate, and starch and the like.

As used herein, the term “tablet glidant” is intended to mean agents used in tablet and capsule formulations to reduce friction during tablet compression. Such compounds include, by way of example and without limitation, colloidal silica, cornstarch, talc, and the like.

As used herein, the term “tablet lubricant” is intended to mean substances used in tablet formulations to reduce friction during tablet compression. Such compounds include, by way of example and without limitation, calcium stearate, magnesium stearate, mineral oil, stearic acid, zinc stearate, and the like.

As used herein, the term “tablet/capsule opaquant” is intended to mean a compound used to render a capsule or a tablet coating opaque. An opaquant may be used alone or in combination with a colorant. Such compounds include, by way of example and without limitation, titanium dioxide and the like.

As used herein, the term “tablet polishing agent” is intended to mean a compound used to impart an attractive sheen to coated tablets. Such compounds include, by way of example and without limitation, carnauba wax, white wax, and the like.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

As used herein, the terms “Polyphenolic compounds,” “Phenolic compounds,” “Polyphenols” “lignans” and “Flavanols” may be used interchangeably to characterized compounds having one or more phenol groups or subcomponent per molecule. Within the general term “polyphenols” are also included the dihydroxy and trihydroxy benzoic acids and the phytoalexins, specifically pinoresinol, oleocanthal, hydrotyrosol, tyrosol, secoiridoids, lignans, gallic acid, resveratrol, hydroxytirosol, and deacetoxy-ligstroside aglycone. Other examples include, catechins flavonoids, polyphenols, Procyanidin polyphenols, flavones, flavanols, flavan-3-ols, proanthocyanidins, procyanidols, procyanins, procyanidins, tannins. The term is also intended to encompass associated derivatives which are additionally substituted, especially those compounds which include substitutions and modification including but not limited to alkylation, methylation, ethylation, alkoxyation, sulphation, malonylation and one or more sugar residues (such as glucose, galactose, arabinose, rhamnose and the like), particularly O-glycosylated compounds so forth. The compounds also include compounds that may be classified into tannins, and phenylpropanoids, e.g., lignins and flavonoids. The compounds may be derived from numerous sources including fruit, fruit skin, plants, grains, nuts, berries, tea, beer, grapes, wine, olive oil, chocolate, cocoa, walnuts, peanuts, yerba mate, fruits and vegetables.

As used herein, the terms “Cancer” means an increase in the number of abnormal cells derived from a given normal tissue or any clinical definition. In addition, it may involve the invasion of adjacent or non-adjacent tissues and/or the lymphatic or blood-borne spread of malignant cells to other sites and/or regional lymph nodes. Furthermore, the term also encompasses hyperplasia, precancerous cells and minor preneoplastic changes.

As used herein, the term “Preventing cancer” means to inhibit the transformation of a cell into an abnormal cell by a carcinogenic agent or agents and/or to inhibit the accumulation of cells expressing cancer-specific genes to a number that creates one or more clinical symptoms associated with cancer.

As used herein, the terms “Treating cancer” and “treatment of cancer” mean to at least partially inhibit the replication of cancer cells, to inhibit the spread of cancer, to decrease tumor size, to lessen or reduce the number of cancerous cells in the body, and to ameliorate or alleviate the symptoms of the disease caused by the cancer. The treatment is considered therapeutic if there is a decrease in mortality and/or morbidity.

As used herein, “Extraction” refers to a technique for separating a mixture of chemical components, wherein the components that are separated have different solubilities, molecular weights, charge, adsorption strengths, ionic strengths or a combination thereof. In some instances, the components separated from a mixture of compounds from plant tissue. A “Solvent extraction” is a type of extraction wherein a mixture of components are separated utilizing the differences in the solubilities and adsorption strengths of the components that are separated. As used herein, “Selective extraction” refers to the process of extracting a class of one or more compounds (e.g., one or more non-acidic compounds) from another class of one or more compounds (e.g., one or more acidic compounds). The skilled artisan will recognize other methods for extracting and separating the compounds of the present invention, see e.g., U.S. Pat. No. 7,198,808, which provides a method for selectively extracting acidic and/or non-acidic compounds from natural material such as plant tissue and is incorporated by reference in its entirety.

The phrases “pharmaceutically” or “pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

In addition, the present invention also includes compounds that are modified and/or substituted, e.g., aglycone or O-glycosides (e.g. with D-glucose, galactose, arabinose, rhamnose etc). The term polyphenols and flavanols is also intended to encompass associated derivatives which are additionally substituted, especially those compounds which include substitutions and modification including but not limited to alkylation, methylation, ethylation, alkoxyation, sulphation, malonylation and one or more sugar residues (such as glucose, galactose, arabinose, rhamnose and the like), particularly O-glycosylated compounds so forth.

In addition, the composition of the present invention may be coated. For example, coated particle for use with the present invention is disclosed in U.S. Pat. No. 4,221,778, in which a selective, prolonged continuous release of pharmacologically active drugs, under conditions such as those encountered in the gastrointestinal tract, is achieved by the application of a diffusion barrier coating to an ion exchange drug-resin complex particle which has been treated with a solvating agent. Another prolonged release formulations from coated drugs may be prepared under circumstances wherein a component of the formulation includes a second ionic substance (e.g., a combination drug, a dye, a dispersing agent or the like) bearing the same ionic charge as the drug on the drug-resin complex by employing the second ionic substance in the ion form of an exchange resin complex. The manufacture of a formulation of any drug for liquid dosage usage requires that the final formulation have the drug dissolved or suspended in a liquid that possess extended shelf-life stability and exhibit no change in active drug dosage level over a period of time and has acceptable taste. Thus, to prepare a liquid formulation of any type drug it may be necessary to employ extenders such as water or syrup, and to add flavors, sweeteners, thickening agents, dyes and the like. To control the dissolution profile of the formulation versus the dissolution profile of the same drug in water, the coated particles may also be included in the presence of ionic substances bearing the same ionic charge as the sustained release drug present in the formulation as a coated drug-resin complex. The presence of ionic substances of opposite charge in the final solution, do not have an effect on the expected dissolution rate and improve the release profile. In fact, the second ionic material need not be coated with the water-permeable diffusion barrier coating.

Wurster coating. Examples of resin drug complexes for rapid release of a drug in 0.1 normal hydrochloric acid (0.1N HCl) dissolution medium (which simulates the fluids of the gastrointestinal tract) include, e.g., an uncoated and untreated Amberlite IRP-69 phenylpropanolamine complex with a 22.5% drug loading released 86.3% of the drug in 1 hour. Some retardation of this rapid release can be obtained by attempting to coat the complex particles, without glycerin pretreatment, with a diffusion barrier coating. The efficiency of the coating on the complex particles can be improved and the release of the drug further slowed by treating the resin particles prior to coating with, e.g., about 15-25% glycerin, resulting in the ability to selectively prolong the release of drugs from drug-resin complexes. While the glycerin may be applied to the drug-resin complex, it may be applied to the resin prior to complexing, as in the case where the resin particles are coated prior to complexing with the drug.

Compositions and formulations for parenteral, intrathecal or intraventricular administration may include sterile aqueous solutions, which may also contain buffers, diluents and other suitable additives such as, but not limited to, penetration enhancers, carrier compounds and other pharmaceutically acceptable carriers or excipients.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. For oral, buccal and sublingual administration, the pharmaceutical composition of the invention may be administered as either solutions or suspensions in the form of gelcaps, caplets, tablets, capsules or powders.

For gelcap preparations, the pharmaceutical formulation may include oils, e.g.: (1) fixed oils, such as peanut oil, sesame oil, cottonseed oil, corn oil and olive oil; (2) fatty acids, such as oleic acid, stearic acid and isostearic acid; and fatty acid esters, such as ethyl oleate, isopropyl myristate, fatty acid glycerides and acetylated fatty acid glycerides; (3) alcohols, such as ethanol, isopropanol, hexadecyl alcohol, glycerol and propylene glycol; (4) glycerol ketals, such as 2,2-dimethyl-1,3-dioxolane-4-methanol; (5) ethers, such as poly(ethylene glycol) 450; (6) petroleum hydrocarbons, such as mineral oil and petrolatum; and (7) water, or with mixtures thereof, with or without the addition of a pharmaceutically suitable surfactant, suspending agent or emulsifying agent.

Adsorption of the complexes or compounds onto the ion exchange resin particles to form the active agent-resin complex is a well-known technique as shown in U.S. Pat. No. 2,990,332 (relevant portions incorporated herein by reference) and demonstrated in the examples herein. In general, the complexes or compounds are mixed with an aqueous suspension of the resin and the complex is then dried. Adsorption of complexes or compounds onto the resin is detected by a change in the pH of the reaction medium.

Liquid Dosage Forms: The liquid dosage formulation and method of this invention may be applied generally to liquid formulations of active agents, which may be prepared conventionally as described herein and, for example, to those liquid formulations contained in commercially-available dosage forms that include the two or more salts of the same active agent. The liquid may be provided as an encapsulated liquid formulation. Examples of commercially available encapsulated liquid formulations for use with the present invention include, e.g., ethchlorvynol, nifedipine, etoposide, digoxin, ranitidine hydrochloride, cyclosporin, calcifediol, ethosuximide, nifedipine, calcitriol and tretinoin.

The dosage form may also include an antioxidant to slow or effectively stop the rate of any autoxidizable material present in the dosage form, particularly if it is in a liquid formulation within, e.g., a gelatin capsule. Representative antioxidants include, e.g., ascorbic acid; alpha tocopherol; ascorbyl palmitate; ascorbates; isoascorbates; butylated hydroxyanisole; butylated hydroxytoluene; nordihydroguiaretic acid; esters of garlic acid having at least 3 carbon atoms comprising a member selected from the group consisting of propyl gallate, octyl gallate, decyl gallate, decyl gallate; 6-ethoxy-2,2,4-trimethyl-1,2-dihydro-quinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes may be about 0.001% to 25% of the total weight of the composition present in the dosage form. Antioxidants are known to the prior art in U.S. Pat. Nos. 2,707,154; 3,573,936; 3,637,772; 4,038,434; 4,186,465 and 4,559,237, relevant portions incorporated herein by reference.

The liquid dosage form may also contain one or more chelating agents to protect the active agent either during storage or when in use. Examples of chelating agents include fpolyacrylic acid, citric acid, edetic acid, disodium edetic acid and the like. The chelating agent may be co-delivered with the active agent in the environment of use to preserve and protect the active agent in situ. Such chelating agents may be combined with the liquid, active agent formulation in the porous particles, or the chelating agents may be incorporated into the drug layer in which the porous particles are dispersed.

The liquid formulation may also include one or more surfactants, e.g., nonionic, anionic and cationic surfactants, or combinations thereof. Examples of nontoxic, nonionic surfactants suitable for forming a liquid-based formulation include, e.g., alkylated aryl polyether alcohols; polysorbates such as polysorbate 80; polyethylene glycol tertdodecyl throether available as; fatty and amide condensate or; aromatic polyglycol ether condensate; fatty acid alkanolamine or sorbitan monolaurate; polyoxyethylene sorbitan esters; sorbitan monolaurate polyoxyethylene; sorbitan mono-oleate polyoxyethylene; polyoxypropylene-polyoxyethylene; polyglycolyzed glycerides such as Labraosol, polyoxyethylated castor oil such as Cremophor and polyoxypropylene-polyoxyethylene-8500.

By way of example, anionic surfactants include, e.g., sulfonic acids and the salts of sulfonated esters such as sodium lauryl sulfate, sodium sulfoethyl oleate, dioctyl sodium sulfosuccinate, cetyl sulfate sodium, myristyl sulfate sodium; sulated esters; sulfated amides; sulfated alcohols; sulfated ethers; sulfated carboxylic acids; sulfonated aromatic hydrocarbons; sulfonated ethers; and the like. Cationic surface active agents for use with liquid formulations, include, e.g., cetyl pyridinium chloride; cetyl trimethyl ammonium bromide; diethylmethyl cetyl ammonium chloride; benzalkonium chloride; benzethonium chloride; primary alkylamonium salts; secondary alkylamonium salts; tertiary alkylamonium salts; quaternary alkylamonium salts; acylated polyamines; salts of heterocyclic amines; palmitoyl carnitine chloride, behentriamonium methosulfate, and the like. Surfactants with be provided generally, from 0.01 part to 1000 parts by weight of surfactant, per 100 parts of the active agent.

It is contemplated that the present invention may be formulated as an “immediate release” and/or a “extended release” or “delayed release”, e.g., freeze dried, rotary dried or spray dried powders; amorphous or crystalline powders; granules, precipitates or particulates. The immediate release active may be either free flowing or compressed.

The pharmaceutical formulation and compounds and complexes may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

Effervescent pharmaceutical formulations are well known in the art and include, generally, an acid such as citric acid or a mono or dihydrogen salt thereof and a carbon dioxide source such as a carbonate or hydrogen carbonate alkali metal salt, such as sodium hydrogen carbonate. The acid and the carbon dioxide source do not react together when dry but combine to release carbon dioxide and an effervescent effect in the presence of water. The effervescent pharmaceutical compositions for use with the present invention may be in the form of a tablet for dissolving in water or a dispersible powder for sprinkling onto water, prior to administration. The acid and the carbon dioxide source are blended together during manufacture of the composition in the absence of water to prevent premature effervescence.

Effervescent pharmaceutical compositions may be in the form of a tablet for dissolving in water or a dispersible powder for sprinkling onto water, prior to administration. The components of the couple are blended together during manufacture of the composition. Suitable pharmaceutical formulations include effervescent tablets and sachets containing water dispersible powders. Effervescent pharmaceutical formulations according to the present invention may be prepared by blending together the granulates formed by roller compaction with other components prior to processing into, e.g., beads. Roller compaction may also be extended to include other components, such as one or more active ingredients and non-active ingredients or excipients such as lubricants, disintegrants, flavors and sweeteners. For capsule, final processing may include introducing the beads into the capsules using an encapsulation machine.

Monosodium citrate and sodium bicarbonate, are blended together and then roller compacted, in the absence of water, to form flakes that are then crushed to give granulates. The granulates are then combined with the active ingredient or drug or salt thereof, conventional beading or filling agents and, optionally, sweeteners, flavors and lubricants. The granules are then filled together under controlled ambient conditions, to form beads or capsules, respectively. The hardness of the final tablets is influenced by the linear roller compaction strength used in preparing the granulates, which are influenced by the particle size of the monosodium hydrogen carbonate and sodium hydrogen carbonate.

Other additives conventionally used in pharmaceutical compositions may be included, which are well known in the art. Such additives include, e.g.: anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

Other additives include, binders (adhesives), i.e., agents that impart cohesive properties to powdered materials through particle-particle bonding, such as matrix binders (e.g., dry starch, dry sugars), film binders (e.g., PVP, starch paste, celluloses, bentonite and sucrose), and chemical binders (e.g., polymeric cellulose derivatives, e.g., carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose.

For certain compositions it may be useful to provide buffering agents (or bufferants), where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

The compositions of the present invention may additionally contain other adjunct components conventionally found in pharmaceutical compositions, at their art-established usage levels. Thus, for example, the compositions may contain additional, compatible, pharmaceutically-active materials such as, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the formulation.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206 1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499 2506 and 46 49, respectively). Other non-antisense chemotherapeutic agents are also within the scope of this invention. Two or more combined compounds may be used together or sequentially.

It is contemplated that the “immediate release” active may be formulated as, e.g., freeze dried, rotary dried or spray dried powders; amorphous or crystalline powders; granules, precipitates or particulates. The immediate release active may be either free-flowing or compressed. The pharmaceutical formulation may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

As used herein, the term “Gastrointestinal inflammation” refers to inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence. “Mucosal layer of the gastrointestinal tract” is meant to include mucosa of the bowel including the small intestine and large intestine, rectum, stomach (gastric) lining, oral cavity, and the like.

As used herein, the term “Chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory inflammation” is also referred to as “chronic gastrointestinal inflammatory diseases” or “chronic gastrointestinal inflammatory conditions”. Chronic gastrointestinal inflammation include, but are not limited to, inflammatory bowel disease, colitis induced by environmental insults such as administration of chemotherapy, radiation therapy and the like, colitis in conditions such as chronic granulomatous disease (Schappi et al. Arch Dis Child. 2001 February; 1984(2):147-151), celiac disease, celiac sprue, food allergies, gastritis, infectious gastritis or enterocolitis, and other forms of gastrointestinal inflammation caused by an infectious agent, and other similar conditions.

As used herein, “Inflammatory bowel disease” or “IBD” refers to any of a variety of diseases characterized by inflammation of all or part of the intestines. Examples of inflammatory bowel disease include, but are not limited to, Crohn's disease and ulcerative colitis.

In certain embodiments, the present invention may be used for treatment and/or prevention of gastrointestinal inflammation such as inflammatory bowel disease. The term “Inflammatory Bowel Disease” is commonly used to refer to a group of related, but distinct, chronic inflammatory conditions affecting the gastrointestinal tract. Abnormal p53 expression is a key early step in colon carcinogenesis rising in the setting of chronic inflammation, and abnormal p53 regulation can be found in chronically inflamed tissues. Inflammatory bowel disease may be Crohn's disease (CD) and ulcerative colitis (UC), both of which are idiopathic chronic diseases occurring with an increasing frequency in many parts of the world. In the United States, more than 600,000 are affected every year. IBD can involve either or both small and large bowel. CD can involve any part of the gastrointestinal tract, but most frequently involves the distal small bowel and colon. It either spares the rectum, or causes inflammation or infection with drainage around the rectum. UC usually causes ulcers in the lower part of the large intestine, often starting at the rectum. Symptoms vary but may include diarrhea, fever, and pain. Patients with prolonged UC are at an increased risk of developing colorectal cancer. Inflammatory bowel disease may also include other disease such as non-ulcerative colitis, carcinomas, polyps, cysts of the colon and/or rectum, or combinations thereof.

In another embodiment, the present invention may be used to reduce, prevent, and/or manage inflammatory bowel disease, related gastrointestinal pathologies, and symptoms thereof. The inflammatory bowel disease may be associated with one or more intestinal conditions. Thus, in certain embodiments, the present invention may also be used to directly or indirectly reduce, prevent, and/or manage intestinal conditions. Examples of intestinal conditions include, but are not limited to, inflammatory bowel disease, ulcerative colitis, indeterminate colitis, infectious colitis, granulomatous enteritis, Crohn's disease, irritable bowel syndrome, infectious diseases of the small and large intestine, pyloric spasm, abdominal cramps, functional gastrointestinal disorders, mild dysenteries, diverticulitis, acute enterocolitis, neurogenic bowel disorders, including the splenic flexure syndrome and neurogenic colon, spastic colitis, cysts, polyps, and carcinoma.

The present invention may modulate one or more tumor suppression genes and in turn modulate the growth of one or more colon carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, hodgkin's disease cells, non-hodgkin's disease cells, multiple myeloma cells, waldenstrom's macroglobulinemia cells, ewing's tumor cells, wilms' tumor cells and combinations thereof.

In one embodiment, the patient may not have cancer, may be undergoing treatment for cancer, or may already have cancer, have cancer but no metastasis, have cancer and a metastatic cancer, have cancer that is in remission, have cancer that is immunosuppressed as a result of undergone anti-cancer therapy, chemotherapy, radiation or a combination thereof prior to administration of the invention.

The present pharmaceutical composition may also be provided in a variety of dosage forms, (e.g., enveloped pharmaceutical, solution, suspension, cream, ointment, lotion, capsule, caplet, softgel, gelcap, elixir, syrup, emulsion, granule, gum, insert, jelly, paste, pastille, pellet, spray, lozenge, disk, magma, poultice, or wafer) and the like and may contain various additives, e.g.: anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, magnesium lauryl sulfate, carrier, anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate, binders, buffering agents, antioxidants, chelating agents, surfactants, colorant, flavorant, sweetening agent, tablet antiadherents, diluent, excipient, opaquant, glidant, lubricant, polishing agent, pharmaceutically acceptable salts and a combination thereof.

In an another embodiment, a total daily dose of a formulation may be used as a dietary supplement is between about 5 mg to about 2000 mg of polyphenol olive extract that may be administered one or more times daily, e.g., two times, three times, four times, daily.

The dosage forms and compositions may comprise any of the forms and compositions known to the skilled artisan. In one embodiment, the sustained release formulation includes olive polyphenol extracts in a tablet, capsule, gel or a liquid-soluble powder form.

In another embodiment, the invention described herein includes the administration of a composition having one or more compounds extracted from a fruit or vegetable, specifically polyphenol extracted from olives as a dietary supplement for the prevention of cancer.

The present invention may be used to treat carcinoma, glioma, astrocytoma, medulloblastoma, craniopharyngioma, ependymoma, pinealoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemia, acute lymphocytic leukemia and acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, chronic lymphocytic leukemia, polycythemia vera, lymphoma, Hodgkin's disease, non-Hodgkin's disease, multiple myeloma, Waldenstrom's macroglobulinemia, and heavy chain disease.

In another embodiment, the polyphenolic composition of the present invention, with or without a pharmaceutical carrier may be used to treat neoplasia in a subject by affecting abnormal cell proliferation, modifying cell apoptosis, inducing cell cycle arrest, decreasing cell proliferation, modulation of epigenetic changes or combinations thereof.

The present invention provides a method of modulating cell proliferation by contacting cells with a pharmaceutical effective amount of the polyphenolic composition extracted from olives. Cell proliferation may be modulated in many ways, some examples include, but not limited to modifying apoptosis, inducting cell cycle arrest, decreasing cell proliferation, modulating epigenetic changes or a combinations thereof.

The present invention provides methods and compositions for treating colorectal cancer cells; however, other cancer cells including but not limited to carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells or combinations thereof may be treated by the polyphenolic composition of the present invention.

Yet in another embodiment, the polyphenolic composition extracted from olives may be taken as a dietary supplement for the treatment or prevention of neoplasia and/or cancer in a subject. In certain embodiments, the polyphenolic compound of the present invention, with and without a pharmaceutical carrier, may be used in affecting neoplasia cells by inducing cell apoptosis and/or inducing tumor suppressor genes.

In addition, the compositions and methods of the present invention may include or be administering in combination with other therapeutic agents, such as anti-cancer drugs, e.g., but are not limited to adriamycin and adriamycin conjugates, mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouacil, floxuridie, cytarabine, mercaptopurine, thioguanine, pentostatin, vinblastine, vincristine, etoposide, teniposide, actinomycin D, daunorubicin, doxorubicin, bleomycin, plicamycin, mitomycin, L-asparaginase, interferon-alpha, cisplatin, carboplatin, mitoxantrone, hydroxyurea, procarbazine, mitotane, aminoglutethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, acetogenins, bullatacin, quassanoids, simalikalactone D, glaucarubolone, and pharmaceutically acceptable derivatives thereof.

The present invention may be used as a dietary or nutritional supplement for the prevention of cancer. In this embodiment, the total daily dose ranges of the polyphenolic compounds (e.g., pinoresinol and/or oleocanthal) for the conditions described herein are generally from about 10 mg to about 800 mg administered in divided doses administered parenterally or orally. A total daily dose is from about 50 mg to about 400 mg of the active extracts.

The present invention relates to a composition and method for preventing and treating cancer and modulating cell proliferation using compositions extracted from pinoresinol-rich extra virgin olive oil. The present invention provides contacting one or more cells with a pharmaceutical effective amount of a polyphenolic composition. The polyphenolic composition includes one or more polyphenolic compounds extracted from one or more plant tissues.

In one embodiment the one or more polyphenolic compounds comprise pinoresinol, oleocanthal, hydrotyrosol, tyrosol, secoiridoid, gallic acid, resveratrol, hydroxytirosol, deacetoxy-ligstroside aglycone, p-HPEA-EDA, 3,4-DHPEA-EA, p-HPEA-EA or mixtures thereof. For example, the one or more polyphenolic compounds may include between 1 mM and 20 mM of each of pinoresinol, oleocanthal or both. More specifically, the one or more plant tissues comprise a tissue from one or more olives comprise one or more Caiazzana olives from the Campania region of Italy.

The one or more polyphenolic compounds affects cell proliferation in a variety of manners including modifying apoptosis, induction of cell cycle arrest, decrease in cell proliferation, or a combination thereof.

In addition to the preventive nature of the present invention, it may also be used to treat cancer cells, precancerous cells, hyperplasia cells, prehyperplasia cells, preneoplastic cells, preneoplastic cells or mixtures thereof. The one or more cells may be selected from the group of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof. The present invention may also be used in conjunction with an antibiotic agent, e.g., doxorubicin, daunorubicin, epirubicin, idarubicin and anthracenedione, mitomycin C, bleomycin, dactinomycin, and plicatomycin.

Colorectal cancer is the fourth commonest cancer in western countries, affecting over one million of new cases globally every year, with nearly 500,000 deaths. The most effective treatment for colorectal cancer is surgical resection, and early stage diagnosis is crucial for a beneficial outcome. To date, colon cancer prevention guidelines have been completely focused on screening and monitoring high risk patients, and chemopreventive strategies have never been formally introduced, largely because of the absence of safe agents with proven efficacy^{1,2,3,47}. At this time, the side effects of currently available chemopreventive drugs do not justify their application across the general population^{1, 2, 3, 5}. For primary prevention purposes, candidate chemopreventive agents should be exceptionally safe, non-expensive, and effective in suppressing the early phases of the disease. The role of diet in modulating colorectal cancer risk has been investigated in the biotechnological industry. A reasonable corollary of this is that natural compounds, which are proven safe over long periods of time and easily accessible through the diet, might represent the ideal candidates as chemopreventive agents.

The Mediterranean diet is associated with beneficial health properties, including lower incidences of cardiovascular disease, age-related cognitive disease, and cancer^{6, 7}. The present invention provides novel chemopreventive candidates extracted from foodstuffs from the diets of geographical locations that enjoy specific health benefits. For example, olive oil is a key ingredient of the Mediterranean diet, representing the main source of fat. In addition to its unsaturated fatty acids, olive oil provides a variety of minor compounds with beneficial properties^{8, 9}. Bioactive compounds present in small quantities in food have been studied for their effects on health, and in particular phenols and polyphenols have been investigated in disease prevention¹⁰. Three classes of phenolic compounds are represented in differing proportions in olive oil: simple phenols (hydrotyrosol and tyrosol), secoiridoids and lignans⁸. Olive oils possess different phenolic profiles according to the variety of olive, and technical conditions used for production^{11,12}. Other polyphenols from this diet, such as gallic acid and resveratrol, have been demonstrated to induce changes in cell cycle dynamics^{13, 14}.

The biological effects of polyphenols are varied and compound-specific¹⁵. For example, olive oils rich in hydroxytirosol have been demonstrated to inhibit cell proliferation and induce apoptosis in human promyelocytic leukemia and in colorectal cancer cell lines^{18, 16, 17}. The dyaldehyde form of one of the secoiridoids, deacetoxy-ligstroside aglycone (also called oleocanthal), has an ability to inhibit COX-1 and COX-2 similar to that seen with ibuprofen^{18, 19}. Lignans are a major antioxidant component of olive oil and have anticancer effects in the breast, lung, skin and colon¹². Anti-oxidant¹² and antiviral properties²⁰ of lignans have been reported, and the similarities in structure between some lignans and estradiol or tamoxifen suggest possible activity against breast cancer²¹.

The present inventors recognizes that Mediterranean diets are associated with lower incidences of many diseases, including colorectal cancer^{6, 7}. This diet is characterized by the consumption of unsaturated fat, and olive oil is its principal source. There is evidence that the quality rather than the quantity of fat may be important in modulating many disease processes. However, in addition to its unsaturated fatty acids, olive oil is the source of several micronutrient components with beneficial properties, such as α-tocopherol, carotenoids, sterols and phenolic compounds⁹. Certain seed oils that are not associated with health-promoting properties are richer in polyunsaturated fat and vitamin E/tocopherol than olive oil, but do not contain the phenolic compounds¹². Thus, the nutritional issues relevant to a healthful diet may be subtle and complex. The present inventors recognize that pinoresinol-rich extracts is capable to decrease cell viability, induce apoptosis and modulate cell cycle dynamics. More importantly, the present invention provides anticancer effects of extracts from extra virgin olive oil involving cancer cells with protein ATM-p53.

In one embodiment of the present invention, pinoresinol-rich extracts, particularly extracts from extra virgin olive oil is show herein to be capable of decrease cell viability, induce apoptosis and modulate cell cycle dynamics in carcinoma cells such as human colorectal cancer cells. In this embodiment, characterization and examination of different olive oils and their chemopreventive mechanisms are demonstrated herein, particularly, phenolic fraction from two different varieties of olive grown in south-central Italy. The two varieties of olive have significantly different phenolic compositions in which pinoresinol is the major phenolic fraction in olives grown from Caiazzana, Italy, and oleocanthal is the main component of olives grown from Ravece, Italy.

In this embodiment, the present invention demonstrates the anticancer effects of phenolic compounds contained in two different varieties of extra virgin olive oil on in vitro models of colorectal cancer. Crude extra virgin olive oil extracts were used rather than isolated phenols, leading to reason that the naturally-occurring family of compounds present in the oil used in the diet have synergistic properties^{8, 15}. Activities of extra virgin olive oil extracts on cell proliferation, apoptosis and cell cycle regulation and specific pathways involved in the regulation of epithelial cell growth are provided herein.

The two extra virgin olive oil preparations used herein were obtained from olives (Olea europaea) of two varieties named Olea europaea Caiazzana and Olea europaea Ravece. Both olive varieties are characteristic of the central-south regions of Italy. Variety of harvest time, extraction methods and compound identification procedures exist in the field of art. For example, in one embodiment, olives were harvested (e.g., in November) and extracted (e.g., within 4 hours) in a continuous extraction plant, using a percolation-centrifugation system with hammer crushers, a centrifugal decanter, and a separator. Unfiltered oils were stored in filled amber glass bottles at room temperature until extraction. Extraction of phenols from virgin olive oil was performed according to the procedure of Vasquez-Roncero²².

Single phenolic components were identified by liquid chromatography-mass spectrometry, as described by Monti et al.²³, using an API-100 single-quadruple mass spectrometer equipped with an atmospheric pressure chemical ionization ion source, and the analyses were performed using a multi-SIM detection system. The molar concentrations of extra virgin olive oil's phenolic compounds used were determined from the sums of all the compounds present in the chromatogram, and quantitated using a pinoresinol calibration curve. Therefore, the concentrations of the phenolic compounds were expressed in pinoresinol equivalents. The skilled artisan will recognize that there are numerous methods of preparing and extracting compound and that the example herein is merely exemplar in nature.

Cell culture and treatments. The present inventors used human colorectal cancer cell lines RKO, SW480, and HCT116 obtained from the American Type Culture Collection. HCT116^{p53-/-} (p53 knock-out) cells were kindly obtained from Bert Vogelstein. RKO and HCT116 cells have microsatellite instability as a consequence of promoter methylation and homozygous mutation in hMLH1, respectively. Both cell lines have wild-type p53. SW480 is a microsatellite stable cell line with inactivating mutations in the p53 gene²⁴. The cells were cultured in Iscove's Modified Dulbecco's Medium (IMDM) supplemented with 10% fetal bovine serum and 100 U/mL penicillin G and 100 μg/mL streptomycin. The cultures were maintained at 37° C. in 5% CO₂. The treatment with extra virgin olive oil phenolic extracts was performed based on the IC50 results obtained, while changing the conditioned media every 48 hours.

Cell viability (MTT assay). Typically, cells were seeded at a density of 3000 cells/well in a 96-well plate. 24 hours later, cells were treated with concentrations ranging from 0-20 μM of extra virgin olive oil phenolic extracts dissolved in methanol. An appropriate amount of methanol was also added to the control wells. After 96 hours of treatment, the cells were incubated with a solution of MTT [3-(4,5-dimethylthiazol-2-yl)-2,5-diphenyltetrazolium bromide] at a concentration of 0.5 μg/μL for 3 hours at 37° C. The cells were lysed in a buffer containing 10% SDS and 0.01N HCl and analyzed after 12 hours of incubation at 37° C. Colored formazan converted from MTT by viable cells was measured at 570 nm by a microplate reader.

Determination of the induction of apoptosis. Late apoptotic events were analyzed by TUNEL assay using the In Situ Cell Death Detection Kit developed from Roche. The TUNEL assay was performed according to the manufacturer's protocol. Briefly, the cells were plated on glass coverslips in 24-well plates at a concentration of 3000 cells/well followed the next day by 96 hours of treatment at the final concentration of 200 nM EVOO phenolic extracts, changing the conditioned media every 48 hours. An equal amount of methanol was used in the untreated, control cells. Pretreatment with DNase I (3000 U/mL in 50 mM Tris-HCl, 1 mg/ml BSA) was used for the positive controls. Apoptotic cells were visualized under an AxioSkop2 multichannel epi-fluorescent microscope and processed by AxioVision software.

Real Time PCR. Real-time PCR was performed to evaluate changes in the transcript levels of the pro-apoptotic gene Bax. After treatment, total RNA was extracted using TRIzol reagent according to the manufacturer's instructions. TaqMan One Step RT-PCR Master Mix (Roche, Branchburg, N.J.) and the TaqMan Gene Expression Assay for Bax were used. One microgram of RNA from each sample was used as the template. GAPDH was used as an endogenous control. The ABI Prism 7000 Sequence Detection System was used for Real-time PCR analysis. Thermal cycling conditions were designed as follows: RNA retro-transcription at 48° C. for 30 minutes followed by an initial denaturation at 95° C. for 10 minutes and 40 cycles of 95° C. for 15 seconds and 60° C. for 1 minute. The relative quantification of gene expression was performed using the Comparative C_{T} Method (ΔΔCt).

Flow Cytometry. The effects of extra virgin olive oil phenolic extracts on cell cycle profiles were evaluated by flow cytometry. Cell cycle distribution was based on an evaluation of the amount of DNA stained with propidium iodide (PI). Cells were plated at a density of 5×10⁵ cells/plate in 100 mm dishes, synchronized by serum deprivation for 48 hours and finally treated with 200 nM of pinoresinol and 10 μM of oleocanthal, with the conditioned media changed every other day, for a total duration of 96 hours. The cells were harvested, resuspended at a density of 5×10⁶ cells/mL in cold PBS, and fixed with 80% ethanol overnight at −20° C. The next day, the cells were washed, resuspended in 300 μl of PBS, incubated with 160 μg/mL of boiled and renatured ribonuclease A (RNase A) for 15 minutes at 37° C. and stained with 80 μg/mL of PI for 30 minutes. DNA content was evaluated by a FACSCalibur flow cytometer. Cell cycle distribution was determined using the ModFit DNA Analysis Software.

Western Blot Analysis. Protein extraction was performed using Ripa combined with 10 μL/mL of phenylmethylsulfonyl fluoride (PMSF) solution, 10 μL/mL of sodium orthovanadate solution and 10 μL/mL of protease inhibitor cocktail. The appropriate amount of lysis buffer was added to each sample and the pellets were sheared with a syringe. The cell lysates were incubated for 1 hour on ice and centrifuged for 10 minutes to obtain clear supernatants. The protein concentration was measured using the BCA protein assay kit. Aliquots of 40 μg of protein were separated on 10% sodium dodecyl sulfate-polyacrylamide gel electrophoresis (SDS-PAGE) gels. The separated proteins were transferred onto a PVDF membrane. Transferred proteins were stained with Ponceau red to confirm successful transfer, followed by blocking with 5% nonfat milk or 5% BSA in TBS-T (50 mM Tris, pH 7.6, 150 mM NaCl, 0.1% Tween-20). Membranes were then probed with the specific primary antibody followed by incubation with the appropriate secondary antibody, including goat anti-mouse IgG-HRP, donkey anti-goat IgG-HRP, and anti-rabbit IgG-HRP. The membranes were visualized using the ECL Plus chemiluminescence system and scanned with a Storm 840 PhosphorImager. Quantification of the bands was performed using ImageQuant 5.2 spot densitometric software. The expression levels of the proteins were normalized to the expression of the housekeeping protein β-actin. The primary antibodies, including anti-cyclin D1 (clone A-12), anti-cyclin E (clone HE 12), anti-cyclin B1 (clone D11), anti-p53 (clone DO-1), anti-p21 (clone F5), anti-GADD45 (C4), anti-14-3-3σ (N14), Chk1 (G4), and Chk2 (B4) were obtained from Santa Cruz Biotechnology Inc. and incubated for 3 hours at room temperature. The primary antibodies anti-phospho p53 (Ser15) and anti-cdc2 were purchased from Cell Signaling Technology, and the anti-ATM (clone 3E8) was purchased from GeneTex and incubated overnight at 4° C. All antibodies were used at a working concentration of 2 μg/mL.

Statistical Analysis. A two-way ANOVA model, including the effects for cell line, dose, and interaction between cell line and dose, was used to evaluate the relationship between cell viability and those effects. The relative amount of Bax gene expression was quantified using the Comparative C_{T} Method (ΔΔC_{T}). A one-way ANOVA approach was used to compare the fold changes among the cell lines, and the Tukey-Kramer method for pair wise comparisons. The Chi-square test was performed to assess the differences in the cell cycle distributions before and after treatment in each cell line, and then a two-way ANOVA including the effects for cell line and treatment was used to assess the treatment effect on G2/M cell cycle checkpoint arrest. A two-sample t-test was used to compare the level of gene expression in the presence of treatment between p53-proficient and p53-deficient cell lines. Significance was assumed for value of p<0.05.

Identification of compounds and their concentration in pinoresinol extract and oleocanthal extract are shown in TABLE 1.

TABLE 1
Main phenolic compounds
in extra virgin olive oil extracts (%)

| | pinoresinol | oleocanthal |
|---|---|---|
| | extract | extract |
| 3,4-DHPEA-EDA | 0 | 22.40% |
| p-HPEA-EDA (oleocanthal) | 6% | 35% |
| 3,4-DHPEA-EA | 16.40% | 16.50% |
| p-HPEA-EA | 15.60% | 7.50% |
| Pinoresinol | 62% | 18.60% |

The main phenolic compound in the pinoresmol extract was found to be pinoresinol (62%), which made up less than 20% of the oleocanthal extract. The oleocanthal extract contained more of the ortho dihydroxy derivatives, thus having higher antioxidant activity than the pinoresinol extract. The oleocanthal extract contained a relatively large amount of p-HPEA-EDA (oleocanthal, the dialdehyde form of decarboxymethyl ligstroside aglycone) that is considered to be the anti-inflammatory moiety, while in the pinoresinol extract, oleocanthal represented only 6% of the total phenolic extract.

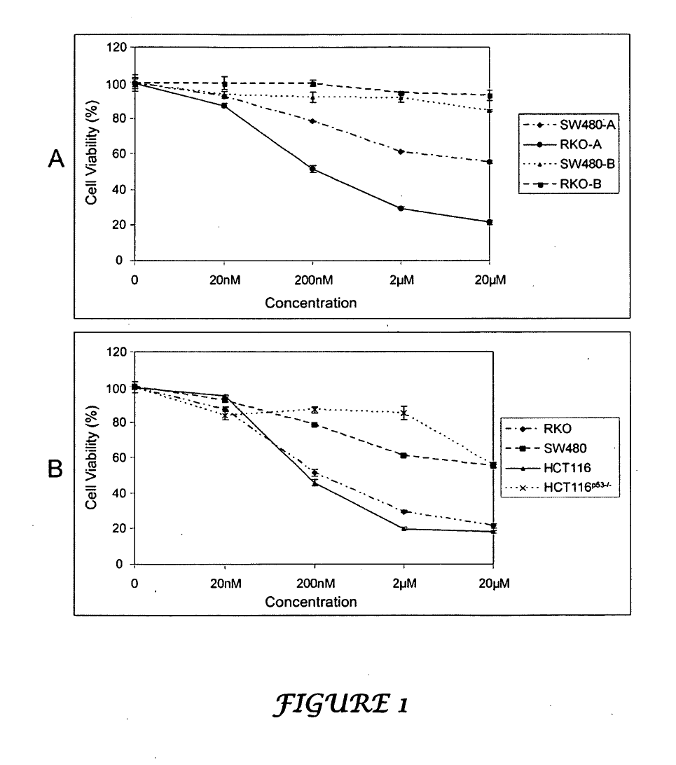

Changes in cell viability were demonstrated using the MTT assay by comparing the effects of the pinoresinol extract and the oleocanthal extract extracts using two model cell lines: RKO and SW480. Cells were treated with the pinoresinol extract and the oleocanthal extract at concentrations ranging from 20 nM to 20 μM for 4 days. Cell viability profile in RKO and SW480 cell lines after 96 hours of the oleocanthal extract (upper curves) and the pinoresinol extract (lower curves) are shown in FIG. 1A. Cell viability is expressed as a ratio of the absorbance between treated cells and untreated controls. Each data point is a mean±SE of three independent assays.

FIGS. 1A and 1B are cell viability plots. FIG. 1A is a plot that demonstrates that only the pinoresinol-rich the pinoresinol extract (the two lower curves) inhibited cellular proliferation in a concentration dependent manner. The p value for overall comparison between the pinoresinol extract and the oleocanthal extract is <0.0001, and a significant difference was seen starting at the second dose. Furthermore, among the pinoresinol extract treated samples, the inhibition of proliferation in RKO (wild-type p53) was significantly greater than with the pinoresinol extract-treated SW480 (inactive p53), starting at 200 nM (p<0.0001). To clarify the differences in viability after the pinoresinol extract treatment by the different genetic profiles of the models used, different cell lines were utilized including the syngeneic pair, HCT116 (p53 proficient) and HCT116^{p53-/-} (p53 knocked out).

FIG. 1B is a plot that demonstrates cell viability after 96 hours of the pinoresinol extract treatment in HCT16^{p53-/-} and SW480 (inactive p53, upper curves), and RKO and HCT116 (wild type p53, lower curves). The effects on the cell viability were significantly more pronounced in the p53 proficient cell lines (p<0.0001), with a decrease in viability of 50% (IC50) in the p53 proficient cells, versus 19%±1.7% in SW480 and 13%±1.5% in HCT116p53−/−.

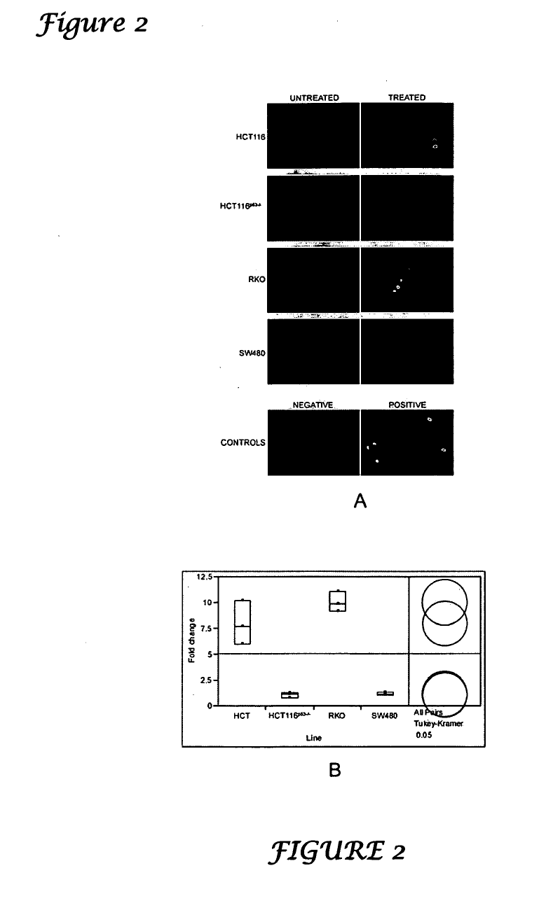

In addition, FIG. 2 demonstrates induced apoptotic response to the pinoresinol extract pinoresinol-rich treatment in p53 proficient cell lines. In FIG. 2A is an image of apoptosis evaluated by TUNEL assay as a measure of DNA fragmentation. The cells were treated for 96 hours with the pinoresinol extract at a concentration of 200 nM. TUNEL positive cells were observed in RKO and HCT 116 (p53 proficient) after treatment, while no staining was detectable in SW480 and HCT116^{p53-/-}.

In FIG. 2B is a plot of the changes in the expression of Bax evaluated by real-time PCR. Each point on the left represents an independent assay, performed in triplicate. The relative amounts of Bax transcripts were quantified using the Comparative C_{T} Method (ΔΔC_{T}). HCT116 and RKO expressed significantly more Bax than HCT116P53-1- and SW480 (p<0.0001). On the right side of FIG. 2B, using a pair-wise Turkey Kramer test, p53 proficient and deficient cell lines were distributed into two statistical different groups: HCT116 and RKO (upper circles) vs. HCT116^{p53-/-} and SW480 (lower circles), p<0.004. In addition, the expression of Bax was evaluated with the treatment of the oleocanthal extract since p53-mediated apoptosis was a specific effect of pinoresinol-rich olive oil. Based on the MTT results, no significant effects on cell viability were detected at concentrations ranging from 200 nM to 20 μM. Moreover, when the cells were treated with the oleocanthal extract at concentrations ranging from 200 nM to 20 μM, no changes in Bax transcripts were detected. The pinoresinol extract treatment caused a decrease of cell viability in RKO and HCT116 cells, together with an increase of apoptosis and prominent G2/M arrest, while no significant changes demonstrated in SW480 and HCT116^{p53-/-}.

The principal difference between these cells is the presence of a functional p53 axis in RKO and HCT116, which is crucial for the regulation of both apoptosis and cell cycle arrest. Cellular stresses and DNA damage typically trigger the p53 tumor suppressor gene to mediate a series of anti-proliferative strategies that preserve genomic fidelity by inducing both cell cycle arrest and apoptosis^{26, 27}. One important link between p53 and apoptosis is based on the transcriptional control of pro-apoptotic members of the Bcl-2 family, such as Bax²⁶. The pinoresinol-rich oil is able to increase apoptosis, as demonstrated by DNA fragmentation and increased Bax transcription, but only in cells with an intact p53 axis.

The present invention also illustrates the effects of the pinoresinol extract treatment on cell cycle progression using flow cytometry. Cell cycle analysis in the pinoresinol extract-treated samples and untreated controls shows that Pinoresinol-rich extract induces G2/M cell cycle arrest selectively in p53 proficient cell lines.

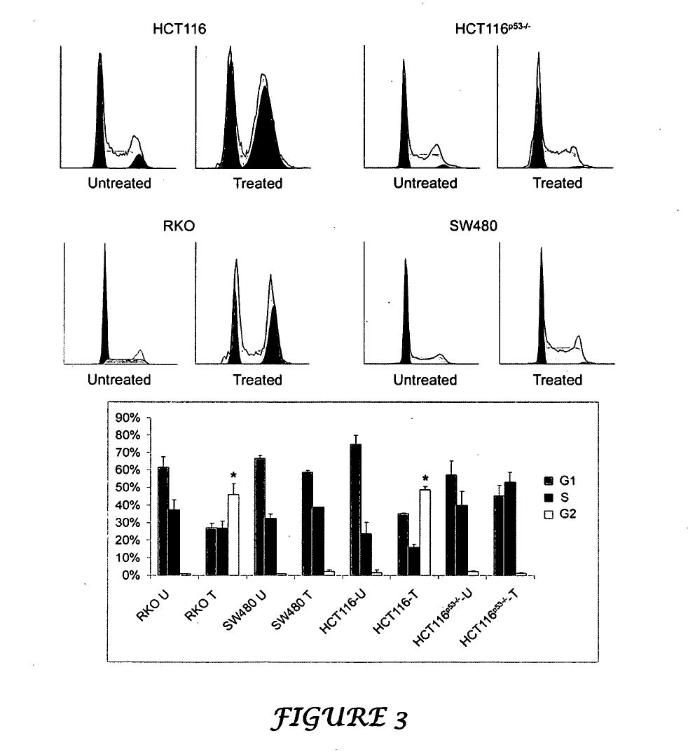

In FIG. 3, a plot of the representative flow cytometry cell cycle profiles for each cell line are shown in the upper panels. First, the cells were synchronized by serum deprivation and subsequently treated every 48 hours, for 96 hours, with 200 nM EVOO-A. In the p53 proficient cell lines, the pinoresinol extract treatment induced a significant arrest in G2/M, with an increase in the G2 population in RKO and HCT116 from 0.96%±0.056 and 2.08%±0.3 to 45%±9.1 and 48%±2.6 respectively (p<0.0001). Corresponding decreases of G1 and S phase cells were observed. No significant changes in the cell cycle profiles were demonstrated in p53-deficient cell lines in response to the pinoresinol extract treatment. Each data point was repeated three times and the distribution of the mean among the different set is shown in the histograms in the lower half of FIG. 3. No changes in the cell cycle profiles were demonstrated after the oleocanthal extract treatment in the two models of colorectal cancer. Taken together, these results illustrates that only pinoresinol-rich olive oil affects apoptosis and cell cycle arrest, and that these effects are exclusively found in the p53 proficient cells.

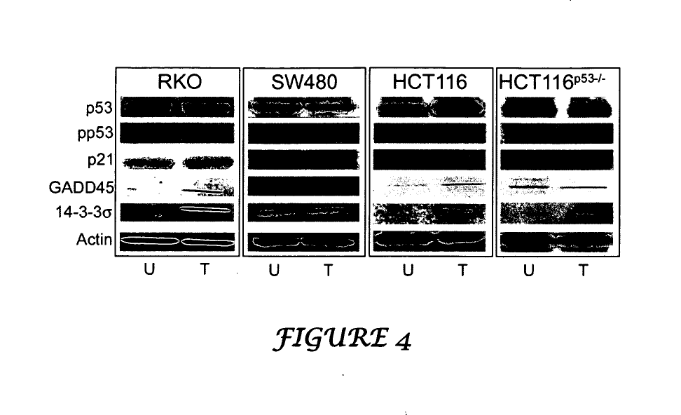

FIG. 4 is an image of a Western blot that demonstrates the treatment of RKO and HCT116 with the pinoresinol extract led to a significant activation and phosphorylation of p53, and the downstream proteins p21^{cip1/waf1}, GADD45 and 14-3-3σ. Using Western Blot analysis, increase of p53 and its activated form, phospho-p53 (Ser15), were observed after treatment specifically in the p53 proficient cell lines, followed by increases in the downstream targets p21^{cip/waf}, GADD 45, and 14-3-3σ (p<0.05). No change in the expression of the p53-regulated axis was observed in SW480 or HCT116^{p53-/-} after treatment.

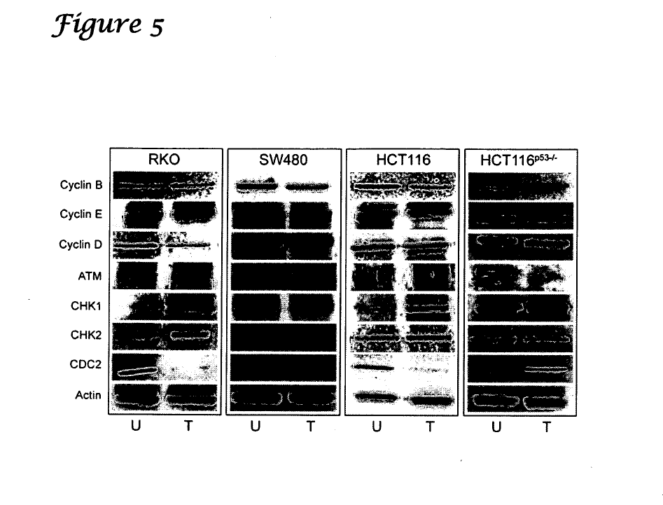

The present invention also demonstrates significant changes in cell cycle checkpoint regulatory proteins such as ATM, Chk1, Chk2, cdc2, and cyclin E after treatment of the pinoresinol extract in RKO and HCT116 cells. FIG. 5 is an image of a Western Blot analysis of protein expression of ATM, Chk1, Chk2 and cdc2 (increased expression) and cyclin E (decreased expression). The pinoresinol extract treatment induced a significant increase of ATM protein levels exclusively in p53 proficient cell lines. Significant changes in Chk1 and Chk2 protein expression were also detected in RKO and HCT116 treated samples. In the p53 deficient cell lines SW480 and HCT116P53, the system was not affected by the treatment. A slight decrease in cyclin B and no change in cyclin D expression was evident in the p53-proficient cell lines. No changes in protein expression were detected after treatment of the p53-deficient cell lines. In RKO and HCT116^{p53-/-}, the activation of these regulatory pathways resulted in a corresponding decrease in cdc2 protein levels (p<0.05). Slight decreases in cyclins B and E, controlled by cdc2, were also observed, while no changes were observed in cyclin D levels.

In terms of cell cycle regulation, p53 can mediate both G1 and G2 phase arrest. For control of the G2 μM checkpoint, p53 is directly controlled by the ATM-ATR genes²⁸⁻³⁰. Pinoresinol-rich olive oil induced G2 cell cycle arrest in p53 proficient cell lines. Inhibitory phosphorylation of cdc2/cyclin B is essential for G2 arrest, and this is regulated by p53-dependent and independent pathways, both of which are downstream of ATM^{29, 30}. The present inventors demonstrated that G2 arrest by pinoresinol-rich olive oil takes place through the up-regulation of ATM-p53 and their downstream pathways (p21^{cip/waf} GADD45 and 14-3-3σ) with a reciprocal decrease in cdc2. In addition, activation of the Chk1 and Chk2 kinases contributes to the G2/M arrest. Chk1 and Chk2 mediate the stabilization of p53 by the phosphorylation of serine 20 in the amino terminal region^{25, 31, 32}, which lead to the reason G2/M arrest was found exclusively in the p53 proficient cells.

Based on the fact that G2/M cell cycle checkpoint arrest occurs only in the p53 proficient cell lines after pinoresinol-rich olive oil treatment, the present invention demonstrates p53 cascade mediated these changes. The activation of the cyclin dependent kinase cdc2 and the cdc2/cyclin B complex are crucial for entry into mitosis. p53 activation depends on ATM status, and three p53 downstream pathways can inhibit the cdc2/cyclin B complex. These involve p21^{cip/waf}, 14-3-3σ and GADD45, respectively. Furthermore, two kinases, Chk1 and Chk2, can mediate G2 arrest by stabilizing p53 through phosphorylation at its amino-terminus²⁵.

The present invention shows that the pinoresinol extract induced G2/M arrest found in RKO and HCT116 was dependent on both the ATM-p53 as well as the ATM-Chk1/Chk2 cascades. The latter system may directly mediate ATM-p53 G2/M arrest, and contribute to cell cycle arrest by enhancing p53 stabilization. Although colorectal cancer cell lines were demonstrated in the above embodiment, the present invention may also be used to modulate and/or affect other cancer cell types, e.g., breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstone tumor, islet cell tumor, primary brain tumor, acute and chronic lymphocyctic and granulocytic tumors, hairy-cell tumor, adenoma, hyperplasia, medullary carcinoma, pheochromocytoma, mucosal neuromas, interstinal ganglioneuromas hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythemia vera, adenocarcinoma, glioblastoma multiforma, acute myeloid leukemia, acute promyelocytic leukemia, acute lymphoblastic leukemia, chronic myelogenous leukemia, myelodysplastic syndrome, lymphomas, malignant carcinomas, epidermoid carcinomas cell lines or any combinations thereof.

The present invention provides a method of treating a variety of types of cancers, include, but are not limited to carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells or any combinations thereof.

The pinoresinol-rich extract of the present invention may further contain additional substance, such as a pharmaceutical agent to form pharmaceutical compositions. Pharmaceutical compositions of the current invention may be prepared by procedures known in the art using well known and readily available ingredients. In addition, the pinoresinol-rich extract may be administered in any currently known method of administration including but not limited to oral dosage forms such as capsules, softgels, tablets, suppositories, injectables, topical formulations or combinations thereof.

For example, the extracted phenolic compounds of the present invention (e.g., pinoresinol-rich extracts, oleocanthal-rich extracts and mixtures thereof) may be mixed with appropriate fillers in relation to the method of administration to be chosen, e.g., omega-3 fatty acid. Omega-3 fatty acids is an ideal choice of vehicle as it serves as limiting bacteria growth and improves the ability for the extracted phenolic compounds of the present invention to be taken up by the cancer cell which needs more fatty acids than normal cells due to their need in building their cell membrane.

The extracted phenolic compounds of the present invention may optionally contain additional substance such as a suitable amount of a physiologically acceptable excipient so as to provide the form for proper administration to the subject. Such physiologically acceptable excipients can be liquids, such as water and oils, including those of petroleum, subject, vegetable, or synthetic origin, such as peanut oil, soybean oil, mineral oil, sesame oil and the like. The physiologically acceptable excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the composition is administered intravenously. Saline solutions and aqueous dextrose and glycerol solutions can also be employed as liquid excipients, particularly for injectable solutions. Suitable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

In certain embodiments, different subjects may be treated by the pinoresinol-rich extract. Accordingly, a subject may be a patient, e.g., a human, suffering from a cancer, or suspected of having a cancer, or at risk for developing a cancer. In some embodiments, the terms “subject” and “patient” may be used interchangeably. A healthy subject may be a human who is not suffering from a cancer or suspected of having a cancer, or who is not suffering from a chronic disorder or condition. A “healthy subject” may also be a subject who is not immunocompromised. Immunocompromised means any condition in which the immune system functions in an abnormal or incomplete manner, for example, a condition, which prevents or reduces a full and normal immune response. Immunocompromisation may be due to disease, certain medications, or conditions present at birth. Immunocompromised subjects may be found more frequently among infants, children, the elderly, individuals undergoing extensive drug or radiation therapy.

In certain embodiments, it is preferable for the pinoresinol-rich extract of the present invention to be combined with traditional, non-traditional, and existing therapies for cancer. Examples include, but not limited to chemotherapy, radiation therapy, surgery, other therapy intended to stimulate the immune system or combinations thereof. Other substance such as nutrients, vitamins and supplements, antioxidants, vitamins A, D, E, C, and B complex; selenium; zinc; co-enzyme Q10, beta carotene, fish oil, curcumin, green tea, bromelain, resveratrol, ground flaxseed, garlic, lycopene, milk thistle, melatonin, cimetidine, indomethacin or any above mentioned combinations may also be administered in conjunction.

In certain embodiments, anti-cancer agent may be combined with the pinoresinol-rich extract. Examples include, but not limited to altretamine, aminoglutethimide, amsacrine, anastrozole, asparaginase, bcg, bicalutamide, bleomycin, buserelin, busulfan, calcium folinate, campothecin, capecitabine, carboplatin, carmustine, chlorambucil, cisplatin, cladribine, clodronate, colchicine, crisantaspase, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, hormones, steroids, steroid synthetic analogs, 17a-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex, antiangiogenics, matrix metalloproteinase inhibitors, VEGF inhibitors, ZD6474, SU6668, SU11248, anti-Her-2 antibodies, EGFR inhibitors, EKB-569, Imclone antibody C225, src inhibitors, bicalutamide, epidermal growth factor inhibitors, Her-2 inhibitors, MEK-1 kinase inhibitors, MAPK kinase inhibitors, P13 inhibitors, PDGF inhibitors, combretastatins, MET kinase inhibitors, MAP kinase inhibitors, inhibitors of non-receptor and receptor tyrosine kinases, inhibitors of integrin signaling, inhibitors of insulin-like growth factor receptors or any combinations thereof.

Yet in another embodiment, the pinoresinol-rich extract of the present invention may be combined with pharmaceutical agents that can alleviate the side effects. Such agents suitable for use include, but are not limited to, anti-emetics, anti-mucositis agents, pain management agents, infection control agents, and anti-anemia/anti-thrombocytopenia agents. Examples of anti-emetics suitable for use herein include, but are not limited to, 5-hydroxytryptamine 3 receptor antagonists, metoclopramide, steroids, lorazepam, ondansetron, cannabinoids, their analogues and derivatives. Examples of anti-mucositis agents suitable for use herein include, but are not limited to, palifermin (keratinocyte growth factor), glucagon-like peptide-2, teduglutide, L-glutamine, amifostin, and fibroblast growth factor 20. Examples of pain management agents suitable for use herein include, but are not limited to, opioids, opiates, and non-steroidal anti-inflammatory compounds. Examples of agents used for control of infection suitable for use herein include, but are not limited to, antibacterials such as aminoglycosides, penicillins, cephalosporins, tetracyclines, clindamycin, lincomycin, macrolides, vancomycin, carbapenems, monobactams, fluoroquinolones, sulfonamides, nitrofurantoins, their analogues and derivatives. Examples of agents that can treat anemia or thrombocytopenia associated with chemotherapy suitable for use herein include, but are not limited to, erythropoietin, and thrombopoietin.

Yet in another embodiment, the pinoresinol-rich extract of the present invention may be combined with antibiotics. Examples include, but not limited to doxorubicin, daunorubicin, epirubicin, idarubicinm anthracenedione, mitomycin C, bleomycin, dactinomycin, plicatomycin, or combinations thereof.

The process of administering the pinoresinol-rich extract, with optional substances may also be varied. Examples include, but not limited to administering orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, or any combinations thereof. It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Vainio H, Miller A B. Primary and secondary prevention in colorectal cancer. Acta Oncol 2003; 42:809-815.
2. Kelloff G J, Lippman S M, Dannenberg A J, Sigman C C, Pearce H L, Reid B J, Szabo E, Jordan V C, Spitz M R, Mills G B, Papadimitrakopoulou V A, Lotan R, Aggarwal B B, Bresalier R S, Kim J, Arun B, Lu K H, Thomas M E, Rhodes H E, Brewer M A, Follen M, Shin D M, Parnes H L, Siegfried J M, Evans A A, Blot W J, Chow W H, Blount P L, Maley C C, Wang K K, Lam S, Lee J J, Dubinett S M, Engstrom P F, Meyskens F L, Jr., O'Shaughnessy J, Hawk E T, Levin B, Nelson W G, Hong W K. Progress in chemoprevention drug development: the promise of molecular biomarkers for prevention of intraepithelial neoplasia and cancer—a plan to move forward. Clin Cancer Res 2006; 12:3661-3697.
3. Souza R F. A molecular rationale for the how, when and why of colorectal cancer screening. Aliment Pharmacol Ther 2001; 15:451-462.
4. American Gastroenterological Association medical position statement: hereditary colorectal cancer and genetic testing. Gastroenterology 2001; 121:195-197.
5. Grau M V, Rees J R, Baron J A. Chemoprevention in gastrointestinal cancers: current status. Basic Clin Pharmacol Toxicol 2006; 98:281-287.
6. Berrino F, Muti P. Mediterranean diet and cancer. Eur J Clin Nutr 1989; 43 Suppl 2:49-55:49-55.
7. Colomer R, Menendez J A. Mediterranean diet, olive oil and cancer. Clin Transl Oncol 2006; 8:15-21.
8. Gill C I, Boyd A, McDermott E, McCann M, Servili M, Selvaggini R, Taticchi A, Esposto S, Montedoro G, McGlynn H, Rowland I. Potential anti-cancer effects of virgin olive oil phenols on colorectal carcinogenesis models in vitro. Int J Cancer 2005; 117:1-7.
9. Perez-Jimenez F, varez de C G, Badimon L, Barja G, Battino M, Blanco A, Bonanome A, Colomer R, Corella-Piquer D, Covas I, Chamorro-Quiros J, Escrich E, Gaforio J J, Garcia Luna P P, Hidalgo L, Kafatos A, Kris-Etherton P M, Lairon D, Lamuela-Raventos R, Lopez-Miranda J, Lopez-Segura F, Martinez-Gonzalez M A, Mata P, Mataix J, Ordovas J, Osada J, Pacheco-Reyes R, Perucho M, Pineda-Priego M, Quiles J L, Ramirez-Tortosa M C, Ruiz-Gutierrez V, Sanchez-Rovira P, Solfrizzi V, Soriguer-Escofet F, de lT-F, Trichopoulos A, Villalba-Montoro J M, Villar-Ortiz J R, Visioli F.
10. Kris-Etherton P M, Hecker K D, Bonanome A, Coval S M, Binkoski A E, Hilpert K F, Griel A E, Etherton T D. Bioactive compounds in foods: their role in the prevention of cardiovascular disease and cancer. Am J Med 2002; 113 Suppl 9B:71S-88S:71S-88S.
11. Bonoli M, Montanucci M, Gallina T T, Lercker G. Fast separation and determination of tyrosol, hydroxytyrosol and other phenolic compounds in extra-virgin olive oil by capillary zone electrophoresis with ultraviolet-diode array detection. J Chromatogr A 2003; 1011:163-172.
12. Owen R W, Mier W, Giacosa A, Hull W E, Spiegelhalder B, Bartsch H. Phenolic compounds and squalene in olive oils: the concentration and antioxidant potential of total phenols, simple phenols, secoiridoids, lignansand squalene. Food Chem Toxicol 2000; 38:647-659.
13. Agarwal C, Tyagi A, Agarwal R. Gallic acid causes inactivating phosphorylation of cdc25A/cdc25C-cdc2 via ATM-Chk2 activation, leading to cell cycle arrest, and induces apoptosis in human prostate carcinoma DU145 cells. Mol Cancer Ther 2006; 5:3294-3302.
14. Tyagi A, Singh R P, Agarwal C, Siriwardana S, Sclafani R A, Agarwal R. Resveratrol causes Cdc2-tyrl5 phosphorylation via ATM/ATR-Chk1/2-Cdc25C pathway as a central mechanism for S phase arrest in human ovarian carcinoma Ovcar-3 cells. Carcinogenesis 2005; 26:1978-1987.
15. Surh Y J. Cancer chemoprevention with dietary phytochemicals. Nat Rev Cancer 2003; 3:768-780.
16. Fabiani R, De B A, Rosignoli P, Servili M, Montedoro G F, Morozzi G. Cancer chemoprevention by hydroxytyrosol isolated from virgin olive oil through G1 cell cycle arrest and apoptosis. Lur J Cancer Prev 2002; 111:351-358.
17. Fabiani R, De B A, Rosignoli P, Servili M, Selvaggini R, Montedoro G F, Di S C, Morozzi G. Virgin olive oil phenols inhibit proliferation of human promyelocytic leukemia cells (HL60) by inducing apoptosis and differentiation. J Nutr 2006; 136:614-619.
18. Beauchamp G K, Keast R S, Morel D, Lin J, Pika J, Han Q, Lee C H, Smith A B, Breslin P A. Phytochemistry: ibuprofen-like activity in extra-virgin olive oil. Nature 2005; 437:45-46.
19. Fogliano V, Sacchi R. Oleocanthal in olive oil: between myth and reality. Mol Nutr Food Res 2006; 50:5-6.
20. Charlton J L. Antiviral activity of lignans. J Nat Prod 1998; 61:1447-1451.
21. Welshons W V, Murphy C S, Koch R, Calaf G, Jordan V C. Stimulation of breast cancer cells in vitro by the environmental estrogen enterolactone and the phytoestrogen equol. Breast Cancer Res Treat 1987; 10: 169-175.
22. Vasquez-Roncero A. Les polyphenols de l'huile d'olive et leur influence sur les characteristiques de l'huile. 25 ed. 1978:21-26.
23. Monti S M, Ritieni A, Sacchi R, Skog K, Borgen L, Fogliano V. Characterization of phenolic compounds in virgin olive oil and their effect on the formation of carcinogenic/mutagenic heterocyclic amines in a model system. J Agric Food Chem 2001; 49:3969-3975.
24. Gayet J, Zhou X P, Duval A, Rolland S, Hoang J M, Cottu P, Hamelin R. Extensive characterization of genetic alterations in a series of human colorectal cancer cell lines. Oncogene 2001; 20:5025-5032.

What is claimed is:

1. A method of modulating cell proliferation comprising the steps of:
contacting one or more cells with an pharmaceutical effective amount of an isolated polyphenolic composition comprising one or more polyphenolic compounds extracted from Olea europaea Caiazzana olives, wherein the one or more polyphenolic compounds reduces cell proliferation.

2. The method of claim 1, wherein the one or more polyphenolic compounds comprise pinoresinol, oleocanthal, hydrotyrosol, tyrosol, secoiridoid, gallic acid, resveratrol, hydroxytirosol, deacetoxy-ligstroside aglycone, p-HPEA-EDA, 3,4-DHPEA-EA, p-HPEA-EA or mixtures thereof.

3. The method of claim 1, wherein the isolated polyphenolic composition is obtained from the oil of the Olea europaea Caiazzana olive.

4. The method of claim 1, wherein the one or more polyphenolic compounds affects cell proliferation by modifying apoptosis, induction of cell cycle arrest, decrease in cell proliferation, or a combination thereof.

5. The method of claim 1, wherein the one or more cells are selected from the group consisting of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof.

6. A polyphenolic composition for the treatment or prevention of colorectal cancer in a subject, comprising:
a pharmaceutical carrier; and
an pharmaceutical effective amount one or more polyphenolic compounds extracted from one or more tissues of an Olea europaea Caiazzana olive or olive oil and reduce the proliferation of one or more colorectal cancer cells, wherein the one or more polyphenolic compounds comprise pinoresinol, oleocanthal, hydrotyrosol, tyrosol, secoiridoids, lignans, gallic acid, resveratrol, hydroxytirosol, deacetoxy-ligstroside aglycone, p-HPEA-EDA, 3,4-DHPEA-EA, p-HPEA-EA or mixtures thereof.

7. The composition of claim 6, wherein the one or more polyphenolic compounds comprises between 100 nM and 20 mM of each of pinoresinol, oleocanthal or a mixture thereof.

8. The composition of claim 6, wherein the one or more polyphenolic compounds comprises greater than about 134 nM of each of pinoresinol, oleocanthal or a mixture thereof.

9. The composition of claim 6, wherein the pharmaceutical composition is in the form of an enveloped pharmaceutical comprising one or more capsules, tablets, pills, liquids, gels or mixtures thereof.

10. The composition of claim 6, wherein the polyphenolic composition is administered orally, intravenously, subcutaneously, parenterally, intraperitoneally, intraarterially, transdermally, sublingually, intramuscularly, transbuccally, intranasally, liposomally, by inhalation, by local delivery, intraadiposally, intraarticularly, intrathecally or a combination thereof.

11. The composition of claim 6, wherein the one or more polyphenolic compounds affects cell proliferation by modifying apoptosis, induction of cell cycle arrest, decrease in cell proliferation, or a combination thereof.

12. A method of anticancer therapy comprising the steps of:
administering to a patient in need of anticancer therapy a pharmaceutical effective amount of a polyphenolic composition comprising one or more polyphenolic compounds extracted from one or more plant Olea europaea Caiazzana olives, wherein the one or more polyphenolic compounds affects the proliferation of one or more cells.

13. The method of claim 12, wherein the one or more cells comprise cancer cells, precancerous cells, hyperplasia cells, prehyperplasia cells, preneoplastic cells, preneoplastic cells or mixtures thereof.

14. The method of claim 12, wherein the one or more polyphenolic compounds comprise pinoresinol, oleocanthal, hydrotyrosol, tyrosol, secoiridoids, lignans, gallic acid, resveratrol, hydroxytirosol, deacetoxy-ligstroside aglycone, p-HPEA-EDA, 3,4-DHPEA-EA, p-HPEA-EA or mixtures thereof.

15. The method of claim 12, wherein the one or more plant tissues comprise a tissue from one or more olives, olive oil and mixtures thereof.

16. The method of claim 12, wherein the one or more plant tissues comprise a tissue from one or more virgin olive oil extracts of Olea europaea Caiazzana olives.

17. The method of claim 12, wherein the polyphenolic composition is administered orally, intravenously, subcutaneously, parenterally, intraperitoneally, intraarterially, transdermally, sublingually, intramuscularly, transbuccally, intranasally, liposomally, by inhalation, by local delivery, intraadiposally, intraarticularly, intrathecally or a combination thereof.

18. The method of claim 12, further comprising administering to the patient a therapeutically effective amount of one or more anticancer treatments selected from the group consisting of radiation therapy, chemotherapy, surgery, immunotherapy, photodynamic therapy, and a combination thereof.

19. The method of claim 12, wherein the one or more cells are selected from the group consisting of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof.

20. The method of claim 12, further comprising administering an antibiotic agent.

21. The method of claim 20, wherein the antibiotic agent is selected from the group consisting of doxorubicin, daunorubicin, epirubicin, idarubicin and anthracenedione, mitomycin C, bleomycin, dactinomycin, and plicatomycin.

22. A pharmaceutical composition for the treatment of neoplasia in a subject, comprising:
a pharmaceutical carrier; and
an pharmaceutical effective amount of one or more polyphenolic compounds extracted from one or more Olea europaea Caiazzana olives, wherein the polyphenolic composition affects abnormal cell proliferation.

23. The composition of claim 22, wherein the one or more polyphenolic compounds comprise pinoresinol, oleocanthal, hydrotyrosol, tyrosol, secoiridoids, lignans, gallic acid, resveratrol, hydroxytirosol, deacetoxy-ligstroside aglycone, p-HPEA-EDA, 3,4-DHPEA-EA, p-HPEA-EA or mixtures thereof.

24. The composition of claim 22, wherein the one or more polyphenolic compounds comprises between 100 nM and 20 mM of each of pinoresinol, oleocanthal or a mixture thereof.

25. The composition of claim 22, wherein the one or more polyphenolic compounds are extracted from a virgin olive oil from one or more Olea europaea Caiazzana olives.

26. The composition of claim 22, wherein the one or more polyphenolic compounds affects cell proliferation by modifying apoptosis, induction of cell cycle arrest, decrease in cell proliferation or a combination thereof.

27. A method for treating neoplasia in a subject, comprising the step of:
administering a pharmaceutical effective amount of a polyphenolic composition to the subject to treat a neoplasia, wherein the polyphenolic composition comprises one or more polyphenolic compounds extracted from one or more Olea europaea Caiazzana olives and affects abnormal cell proliferation in the subject.

28. A dietary supplement for the treatment or prevention of neoplasia in a subject, comprising:
a pharmaceutical carrier; and
an pharmaceutical effective amount of one or more polyphenolic compounds extracted from one or more Olea europaea Caiazzana olives, wherein the polyphenolic composition affects one or more neoplasia cells.

29. A pharmaceutical composition for the treatment of gastrointestinal inflammation in a subject, comprising:
a pharmaceutical carrier; and
an pharmaceutical effective amount of one or more polyphenolic compounds extracted from one or more Olea europaea Caiazzana olives, wherein the polyphenolic composition affects abnormal cell proliferation.

30. The composition of claim 29, wherein the gastrointestinal inflammation is inflammatory bowel disease.

31. The composition of claim 30, wherein the inflammatory bowel disease is Crohn's disease, ulcerative colitis, indeterminate colitis, infectious colitis or combinations thereof.

32. The composition of claim 29, wherein the one or more polyphenolic compounds comprise pinoresinol, oleocanthal, hydrotyrosol, tyrosol, secoiridoids, lignans, gallic acid, resveratrol, hydroxytirosol, deacetoxy-ligstroside aglycone, p-HPEA-EDA, 3,4-DHPEA-EA, p-HPEA-EA or mixtures thereof.

33. The composition of claim 29, wherein the one or more polyphenolic compounds comprises between 100 nM and 20 mM of each of pinoresinol, oleocanthal or a mixture thereof.

34. The composition of claim 29, wherein the one or more olives comprise a virgin olive oil.

35. The composition of claim 29, wherein the one or more polyphenolic compounds affects cell proliferation by modifying apoptosis, induction of cell cycle arrest, decrease in cell proliferation, or a combination thereof.

36. A method for treating gastrointestinal inflammation in a subject, comprising the step of:
administering a pharmaceutical effective amount of a polyphenolic composition to the subject to treat gastrointestinal inflammation, wherein the polyphenolic composition comprises one or more polyphenolic compounds extracted from one or more Olea europaea Caiazzana olives and affects abnormal cell proliferation in the subject.

37. The method of claim 36, wherein the gastrointestinal inflammation is inflammatory bowel disease.

38. The method of claim 37, wherein the inflammatory bowel disease is Crohn's disease, ulcerative colitis, indeterminate colitis, infectious colitis or combinations thereof.

39. A dietary supplement for the treatment or prevention of gastrointestinal inflammation in a subject, comprising:
a pharmaceutical carrier; and
an pharmaceutical effective amount of one or more polyphenolic compounds extracted from one or more Olea europaea Caiazzana olives.
